(19) United States
(12) Patent Application Publication
(10) Pub. No.: US 2020/0316043 A1
(43) Pub. Date: Oct. 8, 2020

(54) PHARMACEUTICAL COMPOSITION COMPRISING CLEVIDIPINE AND PROCESS FOR PREPARATION THEREOF

(71) Applicant: Aurobindo Pharma Ltd., Hyderabad (IN)

(72) Inventors: Amar nath BARIK, Hyderabad (IN); Vikas CHANDEL, Hyderabad (IN); Arvind GANNIMITA, Hyderabad (IN); Nagaprasad VISHNUBHOTLA, Hyderabad (IN); Sivakumaran MEENAKSHISUNDERAM, Hyderabad (IN)

(21) Appl. No.: 16/955,566

(22) PCT Filed: Dec. 18, 2018
(86) PCT No.: PCT/IB2018/060213

§ 371 Date: Jun. 18, 2020

(30) Foreign Application Priority Data
Dec. 20, 2017 (IN) 201741045862

(51) Int. Cl. A61K 31/4422; A61K 9/00; A61K 47/44; A61K 47/24; A61K 47/10; A61K 47/12; A61K 9/107

(52) U.S. Cl. CPC A61K 31/4422; A61K 9/0019; A61K 47/44; A61K 9/107; A61K 47/10; A61K 47/12; A61K 47/24

(57) ABSTRACT

The present invention relates to injectable oil in water pharmaceutical composition comprising effective amount of clevidipine or a pharmaceutically acceptable salt or ester as an active agent and process of preparation thereof. The invention also relates to the use of the emulsion in intravenous administration during surgery and postoperatively in hypertension and for short term treatment of hypertension when oral therapy is not feasible or desirable.

### FIELD OF THE INVENTION

The present invention relates to a pharmaceutical composition comprising clevidipine or a pharmaceutically acceptable salt or ester thereof as an active agent, process of preparation thereof and method of using the same.

This invention relates to an injectable pharmaceutical composition in the form of emulsion of a very short acting, dihydropyridine type calcium channel blocker i.e. clevidipine and a process for preparing such emulsions. The invention also relates to the use of the said emulsion in intravenous administration during surgery for short term treatment of hypertension when oral therapy is not feasible or desirable.

### BACKGROUND OF THE INVENTION

With improved living standards, a diet and lifestyle change in recent years, the incidence of hypertension has been a gradual increasing trend. Hypertension (HTN or HT), also known as high blood pressure (HBP), is a long-term medical condition in which the blood pressure in the arteries is persistently elevated. Affecting more than 30% of the population over 20 years of age, it is one of the most common chronic medical pathologies.

Management of blood pressure is of great importance in many acute clinical situations, e.g. in the majority of patients undergoing cardiac surgery, cerebral surgery, orthopedic surgery or microsurgery, the need to quickly, accurately and safely lower blood pressure to a predetermined level and to maintain it for a certain time after the end of surgery, and then quickly restore the blood pressure to normal levels. In these situations it is important to minimize the volumes given to the patient i.e. administer a concentrated pharmaceutical preparation. Drugs currently used for the above situation are mainly sodium nitroprusside, nitroglycerin and nicardipine. But these therapies are not effective to control blood pressure and the main disadvantage is the risk of having a sodium nitroprusside cyanide poisoning, followed by effects on regional myocardial blood flow in patients suffering from coronary artery disease.

The chemical name of clevidipine is butyroxymethyl methyl 4-(2′,3′dichlorophenyl)-1,4-dihydro-2,6-dimethyl-3,5-pyridinedicarboxylate of the formula C₂₁H₂₃Cl₂NO₆, a molecular weight of 456.3 and having the following chemical formula:

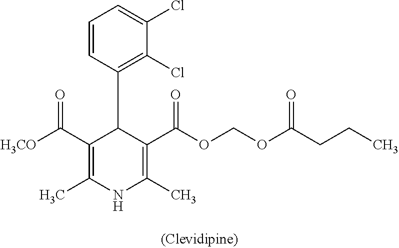 [SMILES: CCCC(=O)OCOC(=O)C1=C(C)NC(C)=C(C(=O)OC)C1c1cccc(Cl)c1Cl]

Clevidipine is further characterized by having low solubility in water and moderate to high solubility in lipids. Oil-in-water emulsions results in better solubility and/or fewer side effects than other conventional solution formulations are utilized. Clevidipine oil-in-water emulsion formulation was first approved by the U.S. FDA in 2008 under the brand name Cleviprex® for intravenous administration in the treatment of acute hypertension, primarily in the emergency room and intensive care unit, and post-operative settings. Oil-in-water emulsions also prevent the lipophilic dihydropyridine compounds from adherence to the plastic infusion sets etc. that are to be used when administrating the compounds.

U.S. Pat. No. 5,856,346 discloses clevidipine compound, as well as suitable pharmaceutical compositions. U.S. Pat. No. 5,739,152 discloses emulsion compositions of clevidipine for intravenous administration comprising a lipid phase, an emulsifier and water or a buffer.

U.S. Pat. No. 8,658,676 describes the pharmaceutical compositions (with EDTA) and preparation of clevidipine which is approved under the brand name Cleviprex®.

The Chinese patent application CN104523590 discloses the parentral pharmaceutical compositions of clevidipine in the form of emulsion. U.S. Pat. No. 5,714,520 describes an oil-in-water emulsion of propofol stabilized by means of a surfactant, and also comprises EDTA (used as an antimicrobial agent).

PCT publication WO1996/024376 discloses a parenteral composition in a solution form comprising of (E)-3-[2-(phenylcarbamoyl) ethenyl]-, 6-dichloroindole-2-carboxylic acid in an isotonic sugar solution containing a water miscible organic solvent for the compound, EDTA required to ensure good solubility in conc. of 0.00768 mg/ml having a pH within the range of 7 to 9.

Several attempts have been made in order to achieve stable injectable emulsion of clevidipine which are described in various patent literature such as CN105497909A; CN105362224A; CN104224735A; CN103211760B.

The low solubility in water and the low compatibility with many excipients specifically with non-ionic emulsion excipients, made it increasingly difficult for preparing the pharmaceutical compositions containing clevidipine.

As mentioned earlier, Cleviprex® was first approved in 2008 which contained soybean oil and egg yolk phospholipids along with glycerin. This composition was not stable for a long period of time. It is recommended that the remaining solution be discarded four hours after opening to avoid microbial contamination requiring the health care providers to continuously provide fresh vials.

In order to overcome this problem, Chiesi reformulated the said composition (Cleviprex®) in 2011 and made it stable by using disodium EDTA and oleic acid. But it has been mentioned in the literature that EDTA inhibits the binding of dihydropyridines like compounds to calcium channel and also binding of dihydropyridines like compounds to brain and cardiac microsomes is inhibited by EDTA indicating both chelating agent and the drug regulate the calcium channels. Also, the specific [³H] nitrendipine (a dihydropyridine calcium channel blocker) binding was reduced by 70-95% due to EDTA treatment to ileal and aortic smooth muscle and cardiac muscle.

Intravenous formulations using disodium EDTA can lead to decline in concentration of calcium since disodium EDTA binds to soluble calcium ion causing reduction of calcium. A rapid decline in blood calcium can lead to muscle spasm. It can cause severe hypocalcaemia, brachiopods, bronchial spasms, seizures and even apnea. Effects on the cardiovascular system, mainly for the conduction block and other arrhythmias, can occur in severe ventricular fibrillation.

Hence, there is still a need to design pharmaceutical composition of clevidipine that have prolonged stability, efficacy and reduced side effects. Inventors of the present invention have endeavored to develop such formulations that are also economical and commercially viable while having none or lower concentration of antimicrobial agent. The compositions of the present invention exhibit excellent storage stability and good tolerance.

The inventors of present invention surprisingly found that clevidipine compositions having low concentrations of antimicrobial agents, antioxidants or preservatives are stable over a prolonged period of time and hence they are less prone to impose electrolyte imbalance and hence avoids side effects such as muscle spasm and/or severe hypocalcaemia.

### SUMMARY OF THE INVENTION

In one aspect, the invention relates to a pharmaceutical composition comprising clevidipine or a pharmaceutically acceptable salt or ester thereof wherein the composition further comprises an amount of antimicrobial agent, preservative or antioxidant or combinations thereof in a sufficient concentration to provide stable composition.

In another aspect, an invention provides oil-in-water emulsion comprising clevidipine or a pharmaceutically acceptable salt or ester thereof and process of preparation thereof.

In another aspect, an invention provides oil-in-water emulsion comprising:

a) clevidipine or a pharmaceutically acceptable salt or ester thereof;
b) 0.00025-0.2% w/v of an antimicrobial agent;
c) optionally other suitable pharmaceutically acceptable excipients.

In another aspect, an invention provides injectable pharmaceutical composition in the form of oil-in-water emulsion comprising:

a) 0.03-0.1% w/v clevidipine or a pharmaceutically acceptable salt or ester thereof;
b) 0.00025-0.2% w/v antimicrobial agent;
c) 10-30% w/v lipid;
d) 0.5-2% w/v emulsifier or co-emulsifier;
e) 1.5-5% w/v tonicity modifier;
f) a pH modifying agent, wherein the pharmaceutical composition is resistant to microbial growth.

In another aspect, an invention provides injectable pharmaceutical composition in the form of oil-in-water emulsion comprising:

a) 0.3-1 mg/ml clevidipine or a pharmaceutically acceptable salt or ester thereof;
b) 0.0025-2 mg/ml antimicrobial agent;
c) 160-240 mg/ml lipid;
d) 5-20 mg/ml emulsifier;
e) 15-50 mg/ml tonicity modifier;
f) a pH modifying agent, wherein the antimicrobial agent is selected from disodium EDTA, sodium sulfite or sodium benzoate and said pharmaceutical composition is resistant to microbial growth.

In another aspect, an invention provides injectable pharmaceutical composition in the form of oil-in-water emulsion comprising:

a) 0.5 mg/ml clevidipine or a pharmaceutically acceptable salt or ester thereof;
b) 0.005-2.0 mg/ml antimicrobial agent selected from disodium EDTA, sodium sulfite or sodium benzoate;
c) 200 mg/ml soybean oil;
d) 12 mg/ml egg yolk phospholipids;
e) 20-45 mg/ml glycerin;
f) a pH modifying agent, wherein the pharmaceutical composition is resistant to microbial growth.

In another aspect, an invention provides injectable pharmaceutical composition in the form of oil-in-water emulsion comprising:

a) 0.05% w/v clevidipine or a pharmaceutically acceptable salt or ester;
b) 0.0005% w/v of disodium EDTA;
c) 20% w/v soybean oil;
d) 1.2% w/v egg yolk phospholipids;
e) 2.0-4.5% w/v glycerin;
f) optionally other suitable pharmaceutically acceptable excipients, wherein the pharmaceutical composition is resistant to microbial growth.

In another aspect, an invention provides injectable pharmaceutical composition in the form of oil-in-water emulsion comprising:

a) 0.05% w/v clevidipine or a pharmaceutically acceptable salt or ester;
b) 0.05-0.2% w/v of sodium sulfite;
c) 20% w/v soybean oil;
d) 1.2% w/v egg yolk phospholipids;
e) 2.0-4.5% w/v glycerin;
f) optionally other suitable pharmaceutically acceptable excipients, wherein the pharmaceutical composition is resistant to microbial growth.

In another aspect, an invention provides injectable pharmaceutical composition in the form of oil-in-water emulsion comprising:

a) 0.05% w/v clevidipine or a pharmaceutically acceptable salt or ester;
b) 0.1% w/v of sodium benzoate;
c) 20% w/v soybean oil;
d) 1.2% w/v egg yolk phospholipids;
e) 2.0-4.5% w/v glycerin;
f) optionally other suitable pharmaceutically acceptable excipients, wherein the pharmaceutical composition is resistant to microbial growth.

In another aspect, an invention provides injectable pharmaceutical composition in the form of oil-in-water emulsion comprising:

a) 0.05% w/v clevidipine or a pharmaceutically acceptable salt or ester;
b) 0.00025-0.009% w/v of disodium EDTA;
c) 20% w/v lipid;
d) 1.2% w/v emulsifier;
e) 2.0-4.5% w/v tonicity modifier;
f) optionally other suitable pharmaceutically acceptable excipients, wherein the pharmaceutical composition is resistant to microbial growth.

In another aspect, an invention provides injectable pharmaceutical composition in the form of oil-in-water emulsion comprising:

a) 0.05% w/v clevidipine or a pharmaceutically acceptable salt or ester;
b) 0.00025-2% w/v of sodium sulfite;
c) 20% w/v lipid;
d) 1.2% w/v emulsifier;
e) 2.0-4.5% w/v tonicity modifier;
f) optionally other suitable pharmaceutically acceptable excipients, wherein the pharmaceutical composition is resistant to microbial growth.

In another aspect, an invention provides injectable pharmaceutical composition in the form of oil-in-water emulsion comprising:

a) 0.05% w/v clevidipine or a pharmaceutically acceptable salt or ester;
b) 0.00025-0.2% w/v of sodium benzoate;
c) 20% w/v lipid;
d) 1.2% w/v emulsifier;
e) 2.0-4.5% w/v tonicity modifier;
f) optionally other suitable pharmaceutically acceptable excipients, wherein the pharmaceutical composition is resistant to microbial growth.

In another aspect, an invention provides injectable pharmaceutical composition in the form of oil-in-water emulsion comprising:

a) 0.5 mg/ml clevidipine or a pharmaceutically acceptable salt or ester;
b) 0.005 mg/ml of disodium EDTA;
c) 200 mg/ml soybean oil;
d) 12 mg/ml egg yolk phospholipids;
e) 22.5 mg/ml glycerin;
f) 0.3 mg/ml oleic acid;
g) optionally other suitable pharmaceutically acceptable excipients, wherein the pharmaceutical composition is resistant to microbial growth.

In another aspect, an invention provides injectable pharmaceutical composition in the form of oil-in-water emulsion comprising:

a) 0.3-1 mg/ml clevidipine or a pharmaceutically acceptable salt or ester;
b) 0.001-0.008 mg/mL of disodium EDTA;
c) 100-300 mg/mL of soybean oil;
d) 6-18 mg/mL of egg yolk phospholipids;
e) 10-35 mg/mL of glycerin;
f) 0.1-0.5 mg/mL of oleic acid
g) Optionally other suitable pharmaceutically acceptable excipients, wherein the pharmaceutical composition is resistant to microbial growth.

In another aspect, an invention provides injectable pharmaceutical composition in the form of oil-in-water emulsion comprising:

a) 0.03-0.1% w/v clevidipine or a pharmaceutically acceptable salt or ester;
b) 0.0001-0.0008% w/v of disodium EDTA;
c) 10-30% w/v of soybean oil;
d) 0.6-1.8% w/v egg yolk phospholipids;
e) 1-3.5% w/v of glycerin;
f) 0.01-0.05% w/v of oleic acid
g) Optionally other suitable pharmaceutically acceptable excipients, wherein the pharmaceutical composition is resistant to microbial growth.

Another aspect of an invention provides the process for the preparation of oil-in-water injectable emulsion composition comprising:
a) Preparing the oil phase consisting of clevidipine to form active phase;
b) Preparing the aqueous phase consisting of an antimicrobial agent in the range of 0.00025-0.2% w/v;
c) Adding oil phase to aqueous phase and adjusting pH;
d) Homogenize under high pressure homogenizer to obtain a suitable dosage form.

An aspect of an invention relates to method to controlling a heart condition such as hypertension by administering injectable oil-in-water emulsion.

In preferred aspects of each embodiment of the invention, the pharmaceutical composition is sterile. In the event of accidental contamination, the pharmaceutical composition will retard the growth of microorganisms.

### DETAILED DESCRIPTION OF THE INVENTION

The term “therapeutically effective amount” is defined to mean the amount or quantity of the active drug (e.g. clevidipine), which is sufficient to elicit an appreciable biological response when administered to the patient.

The term “excipient” means a pharmacologically inactive component such as a solvent, diluent, disintegrant, carrier, or the like. The excipients that are useful in preparing a pharmaceutical composition are generally safe, non-toxic and are acceptable for veterinary as well as human pharmaceutical use. Reference to an excipient includes both one and more than one such excipient.

The term “composition” or “pharmaceutical composition” or “dosage form” or “injectable pharmaceutical composition” as used herein synonymously include dosage forms such as emulsion, solution, lyophilized powder and the like.

In an embodiment, the invention provides injectable oil-in-water emulsion composition comprising effective amount of clevidipine as an active agent and process of preparation thereof.

“Pharmaceutically acceptable excipient(s)” are components that are added to the pharmaceutical composition other than the active ingredient clevidipine. Excipients may be added to facilitate manufacture, enhance stability, enhance product characteristics, enhance patient acceptability etc. Pharmaceutically acceptable excipient(s) includes, but not limited to, one or more lipid, emulsifying agent, surfactant, pH modifier, chelating agent, acidifying agent, solvent, vehicle, oily vehicle, preservative, suspending agent, dispersing agent, and any other excipient known to the art for making pharmaceutical composition. According to the present invention a particular excipient may perform multiple roles in the pharmaceutical composition, for example, it can act both as a preservative and/or as a pH modifier.

In one embodiment, the invention relates to pharmaceutical composition comprising clevidipine or a pharmaceutically acceptable salt or ester thereof wherein composition further comprises an amount of antimicrobial agent sufficient to inhibit growth of the microorganisms.

In another embodiment, an invention provides oil-in-water emulsion comprising clevidipine or a pharmaceutically acceptable salt or ester thereof and process of preparation thereof.

In another embodiment, an invention provides oil-in-water emulsion comprising:

a) clevidipine or a pharmaceutically acceptable salt or ester thereof;
b) 0.00025-0.2% w/v of an antimicrobial agent;
c) optionally other suitable pharmaceutically acceptable excipients.

In another embodiment, an invention provides injectable pharmaceutical composition in the form of oil-in-water emulsion comprising:

a) 0.03-0.1% w/v clevidipine or a pharmaceutically acceptable salt or ester thereof;
b) 0.00025-0.2% w/v antimicrobial agent;
c) 10-30% w/v lipid;
d) 0.5-2% w/v emulsifier;
e) 1.5-5% w/v tonicity modifier;
f) a pH modifying agent, wherein the pharmaceutical composition is resistant to microbial growth.

In another embodiment, an invention provides injectable pharmaceutical composition in the form of oil-in-water emulsion comprising:

a) 0.3-1 mg/ml clevidipine or a pharmaceutically acceptable salt or ester thereof;
b) 0.0025-2 mg/ml antimicrobial agent;
c) 160-240 mg/ml lipid;
d) 5-20 mg/ml emulsifier;
e) 15-50 mg/ml tonicity modifier;
f) a pH modifying agent, wherein the antimicrobial agent is selected from disodium EDTA, sodium sulfite or sodium benzoate and the pharmaceutical composition is resistant to microbial growth.

In another embodiment, an invention provides injectable pharmaceutical composition in the form of oil-in-water emulsion comprising:

a) 0.5 mg/ml clevidipine or a pharmaceutically acceptable salt or ester thereof;
b) 0.005-2 mg/ml antimicrobial agent selected from disodium EDTA, sodium sulfite or sodium benzoate;
c) 200 mg/ml soybean oil;
d) 12 mg/ml egg yolk phospholipids;
e) 20-45 mg/ml glycerin;
f) a pH modifying agent, wherein the pharmaceutical composition is resistant to microbial growth.

In another embodiment, an invention provides injectable pharmaceutical composition in the form of oil-in-water emulsion comprising:

a) 0.05% w/v clevidipine or a pharmaceutically acceptable salt or ester;
b) 0.0005% w/v of disodium EDTA;
c) 20% w/v soybean oil;
d) 1.2% w/v egg yolk phospholipids;
e) 2.0-4.5% w/v glycerin;
f) optionally other suitable pharmaceutically acceptable excipients, wherein the pharmaceutical composition is resistant to microbial growth.

In another embodiment, an invention provides injectable pharmaceutical composition in the form of oil-in-water emulsion comprising:

a) 0.05% w/v clevidipine or a pharmaceutically acceptable salt or ester;
b) 0.05-0.2% w/v of sodium sulfite;
c) 20% w/v soybean oil;
d) 1.2% w/v egg yolk phospholipids;
e) 2.0-4.5% w/v glycerin;
f) optionally other suitable pharmaceutically acceptable excipients, wherein the pharmaceutical composition is resistant to microbial growth.

In another embodiment, an invention provides injectable pharmaceutical composition in the form of oil-in-water emulsion comprising:

a) 0.05% w/v clevidipine or a pharmaceutically acceptable salt or ester;
b) 0.1% w/v of sodium benzoate;
c) 20% w/v soybean oil;
d) 1.2% w/v egg yolk phospholipids;
e) 2.0-4.5% w/v glycerin;
f) optionally other suitable pharmaceutically acceptable excipients, wherein the pharmaceutical composition is resistant to microbial growth.

In another embodiment, an invention provides injectable pharmaceutical composition in the form of oil-in-water emulsion comprising:

a) 0.05% w/v clevidipine or a pharmaceutically acceptable salt or ester;
b) 0.00025-0.009% w/v of disodium EDTA;
c) 20% w/v lipid;
d) 1.2% w/v emulsifier;
e) 2.0-4.5% w/v tonicity modifier;
f) optionally other suitable pharmaceutically acceptable excipients, wherein the pharmaceutical composition is resistant to microbial growth.

In another embodiment, an invention provides injectable pharmaceutical composition in the form of oil-in-water emulsion comprising:

a) 0.05% w/v clevidipine or a pharmaceutically acceptable salt or ester;
b) 0.00025-2% w/v of sodium sulfite;
c) 20% w/v lipid;
d) 1.2% w/v emulsifier;
e) 2.0-4.5% w/v tonicity modifier;
f) optionally other suitable pharmaceutically acceptable excipients, wherein the pharmaceutical composition is resistant to microbial growth.

In another embodiment, an invention provides injectable pharmaceutical composition in the form of oil-in-water emulsion comprising:

a) 0.05% w/v clevidipine or a pharmaceutically acceptable salt or ester;
b) 0.00025-0.2% w/v of sodium benzoate;
c) 20% w/v lipid;
d) 1.2% w/v emulsifier;
e) 2.0-4.5% w/v tonicity modifier;
f) optionally other suitable pharmaceutically acceptable excipients; wherein the pharmaceutical composition is resistant to microbial growth.

In another embodiment, an invention provides injectable pharmaceutical composition in the form of oil-in-water emulsion comprising:

a) 0.5 mg/ml clevidipine or a pharmaceutically acceptable salt or ester;
b) 0.005 mg/ml of disodium EDTA;
c) 200 mg/ml soybean oil;
d) 12 mg/ml egg yolk phospholipids;
e) 22.5 mg/ml glycerin;
f) 0.3 mg/ml oleic acid;
g) optionally other suitable pharmaceutically acceptable excipients, wherein the pharmaceutical composition is resistant to microbial growth.

In another embodiment, an invention provides injectable pharmaceutical composition in the form of oil-in-water emulsion comprising:

a) 0.3-1 mg/ml clevidipine or a pharmaceutically acceptable salt or ester;
b) 0.001-0.008 mg/mL of disodium EDTA;
c) 100-300 mg/mL of soybean oil;
d) 6-18 mg/mL of egg yolk phospholipids;
e) 10-35 mg/mL of glycerin;
f) 0.1-0.5 mg/mL of oleic acid
g) Optionally other suitable pharmaceutically acceptable excipients, wherein the pharmaceutical composition is resistant to microbial growth.

In another embodiment, an invention provides injectable pharmaceutical composition in the form of oil-in-water emulsion comprising:

a) 0.03-0.1% w/v clevidipine or a pharmaceutically acceptable salt or ester;
b) 0.0001-0.0008% w/v of disodium EDTA;
c) 10-30% w/v of soybean oil;
d) 0.6-1.8% w/v egg yolk phospholipids;
e) 1-3.5% w/v of glycerin;
f) 0.01-0.05% w/v of oleic acid
g) Optionally other suitable pharmaceutically acceptable excipients, wherein the pharmaceutical composition is resistant to microbial growth.

Another embodiment of an invention provides the process for the preparation of oil-in-water injectable emulsion composition comprising:
a) Preparing the oil phase consisting of clevidipine or a pharmaceutically acceptable salt or ester to form active phase;
b) Preparing the aqueous phase consisting of an antimicrobial agent in the range of 0.00025-0.2% w/v;
c) Adding oil phase to aqueous phase and adjusting pH;
d) Homogenize under high pressure homogenizer to obtain a suitable dosage form.

The term “clevidipine” as used herein comprises butyroxymethyl methyl 4-(2′,3′dichlorophenyl)-1,4-dihydro-2,6-dimethyl-3,5-pyridinedicarboxylate with a molecular weight of 456.3 g/mol.

Further The term “clevidipine” means all varieties or forms of clevidipine including, but not limited to, all pharmaceutically acceptable salts, esters, amides, isomers, stereo isomers, crystalline and amorphous forms. One particular example is clevidipine butyrate.

The term “clevidipine” or “clevidipine butyrate” as used herein synonymously.

The amount of clevidipine in the composition of the invention may vary depending on the total volume of compositions and the concentration of the other components. The amount of clevidipine according to the invention may be present at a content of from 0.005 to 1% w/v, and preferably from 0.01 to 0.5% w/v and more particularly from 0.03 to 0.1% w/v

In an embodiment, the oil or lipid phase of the composition according to the invention may comprise, for example, any pharmaceutically acceptable oil, preferably triglycerides such as soybean oil, safflower seed oil, olive oil, cottonseed oil, sunflower oil, sesame oil, peanut oil, corn oil, medium chain triglycerides (such as Miglyol® 812 or 810) or triacetin. The oil phase may also be propylene glycol diesters or monoglycerides (such as acetylareal monoglycerides). The oil phase can also be a mixture of said ingredients. The ingredients of the oily phase may be selected by those skilled in the art in order to prepare a composition having the desired properties. The most preferred oil or lipid phase is soybean oil.

In an embodiment, the oily phase of the emulsion according to the invention may be present at a content of from 8 to 35% w/v and preferably from 10 to 30% w/v and more particularly from 15 to 22% w/v.

In an embodiment, the oil phase of the compositions according to the invention advantageously comprise of a suitable surfactant-emulsifier. Emulsifiers are compounds which are capable of improving the wetting of the drug and/or enhancing the dissolution. Suitable emulsifiers include, but are not limited to, propylene glycol mono- and di-fatty acid esters, polyoxyethylene sorbitan fatty acid esters, polyoxyethylene fatty acid esters, polyoxyethylene-polyoxypropylene co-polymers and block co-polymers, salts of fatty alcohol sulphates, sorbitan fatty acid esters, esters of polyethylene-glycol glycerol ethers, oil and wax based emulsifiers, glycerol monostearate, glycerine sorbitan fatty acid esters and phospholipids, preferably phospholipids extracted from egg yolk or soybean, synthetic phosphatidyl cholines or purified phosphatidyl cholines from vegetable origin. Hydrogenated derivatives can also be used, such as phosphatidyl choline hydrogenated (egg) and phosphatidyl choline hydrogenated (soya). The amount of phospholipid emulsifier in the compositions of the present invention can vary depending on the total overall volume of the composition and the concentration of the other components. The emulsifier can also be a mixture of said ingredients. The most preferred emulsifier is egg lecithin. The emulsifier in the emulsion according to the invention may be present at a content of from up to 5% w/v, preferably from about 0.3 to 2% w/v, and more particularly from about 0.5 to 1.5% w/v.

In an embodiment, the oil phase of the composition according to the invention may further comprise a co-emulsifier, a second pharmaceutically acceptable surfactant, wherein the co-emulsifier is selected from the group consisting of synthetic nonionic surfactants such as poloxamers (for example Poloxamer 188 and 407), Cremophor™, poloxamines, polyoxyethylene stearates, polyoxyethylene sorbitan fatty acid esters or sorbitan fatty acid esters, derivatives of tocopherol such as tocopherol PEG succinate, long chain fatty acids such as oleic acid, stearic acid, palmitic acid, bile acids such as cholic acid and deoxycholic acid or surface active derivatives, and pharmaceutically acceptable salts thereof An exemplary co-emulsifier is oleic acid. The co-emulsifier in the emulsion according to the invention may be present at a content of from about 0.005 to 2% w/v, and preferably from about 0.01 to 2% w/v and more particularly from about 0.01 to 1.0% w/v. In particular embodiments, the amount of surfactant in the composition will be about 0.03% w/v.

In an embodiment, the aqueous phase of the composition according to the invention may comprise tonicity modifying agent to make the formulation isotonic with blood. Suitable tonicity modifiers include but are not limited to glycerol, sorbitol, xylitol, mannitol, dextrose, glucose, polyethylene glycol, propylene glycol, sucrose, inorganic salts such as sodium chloride and lactose. The terms “tonicity modifier” and “isotonicity adjuster” are used herein interchangeably. Preferably, the tonicity modifying agent is glycerin. The amount of tonicity modifier used in the emulsions of the present invention may vary from about 1 to 5% w/v, preferably from about 1.5 to 4.5 and more particularly from about 2-4.5% w/v.

In an embodiment, the aqueous phase of the composition according to the invention may further comprise of chelating agents or antimicrobial agents. The chelating agents used to form stable pharmaceutical compositions and dosage forms for their antimicrobial, antioxidant and preservative activity include, but are not limited to Ethylene Diaminetetraacetic acid (EDTA), disodium EDTA, calcium disodium edetate, trisodium EDTA. Other antimicrobial agents include but not limited to chlorhexidine, benzoic acid, sorbic acid, benzyl alcohol, sodium citrate, sodium benzoate, chlorbutanol or a combination thereof. More preferably, the antimicrobial agent is disodium edetate (EDTA) or sodium citrate, or sodium benzoate or sodium sulfite or combination thereof. The amount of the antimicrobial agent in the composition, will generally range from about 0.0001 to 0.2% w/v or from about 0.00025 to 0.2% w/v. In particular embodiments, the amount of the antimicrobial agent in the composition will be about 0.0001, 0.00025, 0.0005, 0.0007, 0.0008, 0.0009, 0.05, 0.1 or 0.2% w/v. Where a chelating agent is used as the antimicrobial agent, the amount of chelating agent in the composition will generally range from about 0.0001 to 0.2% w/v or from about 0.00025% to 0.0009% w/v. The antimicrobial agent may be used alone or in combination with other antimicrobial agents.

In an embodiment, the composition of the present invention may additionally comprise of an antioxidant having preservative activity to restrict the formation of the related substances in the composition including, but are not limited to, sodium ascorbate, cysteine hydrochloride, sodium bisulfite, sodium metabisulfite, sodium sulfite, ascorbyl palmitate, butylated hydroxyanisole (BHA), butylated hydroxytoluene (BHT), propyl gallate, sodium citrate, ascorbic acid esters. The amount of the antioxidant in the composition generally ranges from about 0.01 to 1.0% w/v, preferably about 0.05 to 1.0% w/v, and more particularly from about 0.05 to about 0.5% w/v.

The amount of water in the compositions of the present invention, such as water-for-injections, is used to make up the volume to 100% w/v and can vary depending on the total overall volume of the composition and the concentration of the other components.

In an embodiment, the pH adjusting agent is selected from the group consisting of sodium hydroxide, potassium hydroxide, magnesium hydroxide, sodium carbonate, sodium linoleate, sodium oleate, Tris, potassium carbonate, potassium linoleate, potassium oleate, alone or in combination thereof. The pharmaceutical compositions of the present invention will have a pH that ranges from about 6.0 to about 8.8. In particular embodiments, the pH ranges from about 6.5 to 8.0. In some embodiments, the pH is 6.2, 6.5, 6.75, 7.0, or 7.5.

Parenteral modes of administration include intradermal, subcutaneous (s.c., s.q., sub-Q, Hypo), intramuscular (i.m.), intravenous (i.v.), intraperitoneal (i.p.), intra-arterial, intramedulary, intracardiac, intra-articular (joint), intrasynovial (joint fluid area), intracranial, intraspinal, and intrathecal (spinal fluids) without limitation. Any known device useful for parenteral injection or infusion of drug compositions can be used to effect such administration.

The sterile composition of the invention can be dissolved or suspended in any of the commonly used sterile intravenous fluids and administered by infusion including but not limited to physiological saline, phosphate buffered saline, 5% dextrose in water or Ringer's™ solution. The parenteral dosage form of compositions of the present invention can also be a ready-to-use solution in sterile sealed vials, hermetically sealed ampoules or in sterile pre-filled syringes.

In an embodiment, an invention provides method to controlling the blood pressure by administering injectable pharmaceutical composition of clevidipine or a pharmaceutically acceptable salt or ester thereof to a subject in need of such treatment.

The following examples serve to illustrate the embodiments of the present invention. However, they do not intend to limit the scope of the invention. It is obvious to those skilled in the art to find out the composition for other dosage forms and substitute the equivalent excipients as described in this specification or with the one known to the industry.

TABLE 1

| | | Example 1 | Example 2 | Example 3 |
|---|---|---|---|---|
| No | Ingredients | Qty in mg/mL | | |
| 1 | Clevidipine Butyrate | 0.5 | 0.5 | 0.5 |
| 2 | Soybean oil | 200 | 200 | 200 |
| 3 | Glycerin | 40 | 22.5 | 22.5 |
| 4 | Oleic acid | 0.3 | — | 0.3 |
| 5 | Purified egg yolk | 12 | 12 | 12 |
| | Phospholipid | | | |
| 6 | Sodium Hydroxide | Adjust to pH | Adjust to pH | Adjust to pH |
| | | 6.0-8.0 | 6.0-8.0 | 6.0-8.0 |
| 7 | Water for Injection | Q.s to 1 mL | Q.s to 1 mL | Q.s to 1 mL |

q.s.: Quantity sufficient

Manufacturing Process:

Preparation of Aqueous Phase:

i. Weigh and transfer the batch quantity of water for injection (WFI) in SS container and heat up to 50° C.-70° C. A part quantity of water for injection was kept aside for rinsing.
ii. The batch quantity of glycerin was added to step-i under continuous stirring and mixed well to form clear aqueous phase.

Preparation of Oil Phase:

iii. The batch quantity of soybean oil was weighed and transferred into another SS vessel and heated up to 50° C.-70° C. under stirring followed by addition of batch quantity of Clevidipine (API).
iv. The batch quantities of phospholipids were added to the step-iii followed by optionally the addition of oleic acid, under continuous stirring.

Emulsification:

v. Oil phase was added to the aqueous phase while maintaining the temperature around 60° C.
vi. The pH of coarse emulsion was adjusted with sodium hydroxide (pH range: 6.0-8.0).
vii. The volume of emulsion in step-vi was made up with WFI and the pH was checked again.

Homogenization:

viii. The bulk emulsion was homogenized under High pressure homogenizer.

Filling and Packaging:

ix. The homogenized emulsion was filtered and filled into vials (50/100 mL) with stopper followed by sealing.

TABLE 2

| | | Example 4 | Example 5 | Example 6 |
|---|---|---|---|---|
| No | Ingredients | Qty in mg/mL | | |
| 1 | Clevidipine Butyrate | 0.5 | 0.5 | 0.5 |
| 2 | Soybean oil | 200 | 200 | 200 |
| 3 | Glycerin | 22.5 | 22.5 | 22.5 |
| 4 | Oleic acid | 0.3 | 0.3 | 0.3 |
| 5 | Purified egg yolk | 12 | 12 | 12 |
| | Phospholipid | | | |
| 6 | Disodium EDTA | — | — | 0.005 |
| 7 | Sodium Sulfite | 2.0 | — | — |
| 8 | Sodium Benzoate | — | 1.0 | — |
| 9 | Sodium Hydroxide | Adjust to pH | Adjust to pH | Adjust to pH |
| | | 6.0-8.0 | 6.0-8.0 | 6.0-8.0 |
| 10 | Water for Injection | Q.s to 1 mL | Q.s to 1 mL | Q.s to 1 mL |

q.s.: Quantity sufficient

Manufacturing Process:

Preparation of Aqueous Phase:

i. Weigh and transfer the batch quantity of water for injection (WFI) in SS container and heat up to 50° C.-70° C. A part quantity of water for injection was kept aside for rinsing.
ii. The batch quantity of glycerin and the antimicrobial agent were added to step-i under continuous stirring and mixed well to form clear aqueous phase.

Preparation of Oil Phase:

iii. The batch quantity of soybean oil was weighed and transferred into another SS vessel and heated up to 50° C.-70° C. under stirring followed by addition of batch quantity of Clevidipine (API).
iv. The batch quantities of phospholipids were added to the step-iii followed by the addition of oleic acid under continuous stirring.

Emulsification:

v. Oil phase was added to the aqueous phase while maintaining the temperature around 60° C.
vi. The pH of coarse emulsion was adjusted with sodium hydroxide (pH range: 6.0-8.0).
vii. The volume of emulsion in step-vi was made up with WFI and the pH was checked again.

Homogenization:

viii. The bulk emulsion was homogenized under High pressure homogenizer.

Filling and Packaging:

ix. The homogenized emulsion was filtered and filled into vials (50/100 mL) with stopper followed by sealing.

TABLE 3

| | | Example 7 | Example 8 | Example 9 |
|---|---|---|---|---|
| No | Ingredients | Qty in mg/mL | | |
| 1 | Clevidipine Butyrate | 0.5 | 0.5 | 0.5 |
| 2 | Soybean oil | 200 | 200 | 200 |
| 3 | Glycerin | 22.5 | 22.5 | 22.5 |
| 4 | Oleic acid | 0.3 | 0.3 | 0.3 |
| 5 | Purified egg yolk | 12 | 12 | 12 |
| | Phospholipid | | | |
| 6 | Disodium EDTA | — | — | 0.007 |
| 7 | Sodium Sulfite | 0.5 | — | — |
| 8 | Sodium Benzoate | — | 1.2 | — |
| 9 | Sodium Hydroxide | Adjust to pH | Adjust to pH | Adjust to pH |
| | | 6.0-8.0 | 6.0-8.0 | 6.0-8.0 |
| 10 | Water for Injection | Q.s to 1 mL | Q.s to 1 mL | Q.s to 1 mL |

q.s.: Quantity sufficient

Manufacturing Process:

Same as mentioned under example 4, 5 and 6.

TABLE 4

| | | A | B | C |
|---|---|---|---|---|
| No | Ingredients | Qty in mg/mL | | |
| 1 | Clevidipine Butyrate | 0.3-1 | 0.3-1 | 0.3-1 |
| 2 | Soybean oil | 160-240 | 160-240 | 160-240 |
| 3 | Glycerin | 1.5-5 | 1.5-5 | 1.5-5 |
| 4 | Oleic acid | 0.1-1 | 0.1-1 | 0.1-1 |
| 5 | Purified egg yolk | 5-20 | 5-20 | 5-20 |
| | Phospholipid | | | |
| 6 | Disodium EDTA | — | — | 0.0025-0.009 |
| 7 | Sodium Sulfite | — | 0.5-2 | — |
| 8 | Sodium Benzoate | 0.25-1 | — | — |
| 9 | Sodium Hydroxide | pH 6-8 | pH 6-8 | pH 6-8 |
| 10 | Water for Injection | q.s. 1 ml | q.s. 1 ml | q.s. 1 ml |

Manufacturing Process:

Same as mentioned under example 4, 5 and 6.

Stability Study:

Stability study was conducted on the composition stated in example 6 under two conditions i.e. at 25° C./60% RH, Inverted (Accelerated), at 2-8° C., Inverted (refrigeration/storage condition).

Samples were analyzed to measure assay of Clevidipine, assay of Disodium Edetate Dihydrate, impurities, pH, globule size. The product was found to be stable for six months at each of the above conditions. The details of the stability study of Example 6 is provided in table 5 & 6 below.

TABLE 5
Stability data of example 6 (fill volume 100 ml)

| | | 2-8° C., | | 25° C./60% RH, | |
|---|---|---|---|---|---|
| | | Inverted | | Inverted | |
| Test | Specification | Initial | 6 month | Initial | 6 month |
| Assay of Clevidipine | 90.0-110.0% | 96 | 92 | 96 | 98 |
| Assay of Disodium EDTA | 50-120% | 90 | 81 | 90 | 80 |
| Related Substances | | | | | |
| Total Impurities | NMT 3% | 0.13 | 0.45 | 0.13 | 0.46 |
| Free Fatty acid content | NMT 14 mmol/L | 2.8 | 2.63 | 2.8 | 1.62 |
| pH | Between 6.0 & 8.0 | 7.02 | 6.53 | 7.02 | 6.5 |
| Globule Size (Z average in nm) | NMT 500 nm | 244.9 | 251.8 | 244.9 | 244.5 |

NMT: Not more than

TABLE 6
Stability data of example 6 (fill volume 50 ml)

| | | 2-8° C., | | 25° C./60% RH | |
|---|---|---|---|---|---|
| | | Inverted | | Inverted | |
| Test | Specification | Initial | 6 month | Initial | 6 month |
| Assay of Clevidipine | 90.0-110.0% | 98 | 98 | 98 | 100 |
| Assay of Disodium EDTA | 50-120% | 82 | 78 | 82 | 80 |
| Related Substances | | | | | |
| Total Impurities | NMT 3% | 0.75 | 0.88 | 0.75 | 1.0 |
| Free Fatty acid content | NMT 14 mmol/L | 1.52 | 1.30 | 1.52 | 1.79 |
| pH | Between 6.0 & 8.0 | 7.21 | 6.82 | 7.21 | 7.29 |
| Globule Size (Z average in nm) | NMT 500 nm | 275.3 | 289.4 | 275.3 | 244.5 |

NMT: Not more than

We claim:

1. (canceled)

2. A stable injectable pharmaceutical composition comprising:
a) 0.3-1 mg/mL clevidipine butyrate or a pharmaceutically acceptable salt thereof;
b) 0.001-2.0 mg/mL of antimicrobial agent;
c) 100-300 mg/mL of soybean oil;
d) 6-18 mg/mL of egg yolk phospholipids;
e) 10-35 mg/mL of glycerin;
f) 0.1-0.5 mg/mL of oleic acid
Wherein antimicrobial agent consist of disodium EDTA, sodium sulfite, sodium benzoate or combination thereof.

3. The injectable pharmaceutical composition as claimed in claim 2, comprising antimicrobial agent. in a concentration range of 0.001, 0.0025, 0.003, 0.004, 0.005, 0.006, 0.007, 0.25, 0.50, 0.75, 1.0, 1.20, 1.50 or 2.0 mg/mL.

4. (canceled)

5. The injectable pharmaceutical composition as claimed in claim 2, wherein sodium hydroxide is used as a pH adjusting agent.

6. The injectable pharmaceutical composition as claimed in claim 2, which has a pH of about 6.0 to about 8.0.

7. The injectable pharmaceutical composition as claimed in claim 2, wherein the injectable pharmaceutical composition is in the form of oil-in-water emulsion.

8. (canceled)

9. (canceled)

10. The injectable pharmaceutical composition as claimed in claim 2, wherein the injectable pharmaceutical composition is used in reduction of blood pressure.

11. An injectable pharmaceutical composition comprising:
a) 0.5 mg/mL clevidipine butyrate or a pharmaceutically acceptable salt thereof;
b) 0.001-0.009 mg/mL of disodium EDTA;
c) 200 mg/mL of soybean oil;
d) 12 mg/mL of egg yolk phospholipids;
e) 22.5 mg/mL of glycerin;
f) 0.3 mg/mL of oleic acid
Wherein compositions contains not more than 1.5% of total impurities by weight relative to clevidipine after storage for 6 months at 2-8° C. or 25° C./60% relative humidity.

12. An injectable pharmaceutical composition comprising:
a) 0.05% w/v clevidipine butyrate or a pharmaceutically acceptable salt thereof;
b) 0.0001-0.0007% w/v of disodium EDTA;
c) 20% w/v of soybean oil;
d) 1.2% w/v of egg yolk phospholipids;
e) 2.22-2.27% w/v of glycerin;
f) 0.03% w/v of oleic acid
Wherein compositions contains not more than 1.5% of total impurities by weight relative to clevidipine after storage for 6 months at 2-8° C. or 25° C./60% relative humidity.

13. The pharmaceutical composition according to claim 11, wherein the composition contains 0.001, 0.0025, 0.0040, 0.005, 0.006 or 0.007 mg/mL of disodium EDTA.

14. The pharmaceutical composition according to claim 12, wherein the composition contains 0.0001, 0.00025, 0.00040, 0.0005, 0.0006 or 0.0007 mg/mL of disodium EDTA.

15. The pharmaceutical composition according to claim 2 wherein the stability of the said composition is such that at least 90% amount of clevidipine is present in the composition after storage for 6 months at 2-8° C. or 25° C./60% relative humidity.

16. The pharmaceutical composition according to claim 2 wherein the free fatty acid content of the composition is not more than 14 mmol/L, preferably not more than 4 mmol/L, and more preferably between 1-3 mmol/L after storage for 6 months at 2-8° C. or 25° C./60% relative humidity.

17. The pharmaceutical composition according to claim 2 wherein the composition having globule size of not more than 500 nm, preferably 150-350 nm and more preferably between 230-280 nm after storage for 6 months at 2-8° C. or 25° C./60% relative humidity.

18. The method for preparing the pharmaceutical composition according to claim 2 comprising the steps of:
a) Preparation of oil phase comprising heating soybean oil at 50-70° C. and then adding egg yolk phospholipid followed by oleic acid under stirring and then add clevidipine as a slurry using oil phase with stirring.
b) Preparation of aqueous phase comprising heating water for injection up to 50-70° C. and then adding EDTA followed by glycerine under stirring.
c) Add oil phase to aqueous phase at 60-70° C. under stirring and then adjust pH between 6-8 using sodium hydroxide.

19. The method for preparing the pharmaceutical composition according to claim 18 wherein each phase is heated at 55-70° C. more specifically 60-70° C. and preferably 65-70° C.
